(19) United States
(12) Patent Application Publication
(10) Pub. No.: US 2006/0258906 A1
(43) Pub. Date: Nov. 16, 2006

(54) Systems and methods to facilitate endoscopic

(76) Inventor: Kenneth F. Binmoeller, Rancho Santa Fe, CA (US)

Correspondence Address: PRESTON GATES ELLIS & ROUVELAS MEEDS LLP, 1735 NEW YORK AVENUE, NW, SUITE 500, WASHINGTON, DC, 20006, US

(21) Appl. No.: 11/434,130

(22) Filed: May 16, 2006

Related U.S. Application Data
(60) Provisional application No. 60/681,014, filed on May 16, 2005.

(51) Int. Cl. A61B 1/00

(52) U.S. Cl. 600/114; 600/121

(57) ABSTRACT

Disclosed herein are devices, systems and methods for use with endoscopic instruments. More specifically, the presently described devices, systems and methods utilize overtube devices for use with endoscopic instruments that allow more diagnostic and therapeutic interventions than previously possible.

### CROSS REFERENCE TO RELATED APPLICATION

This application claims priority under 35 U.S.C. § 119 to U.S. Provisional Patent Application Ser. No. 60/681,014, filed on May 16, 2005, the entire contents of which are expressly incorporated by reference herein.

### FIELD OF THE INVENTION

The devices, systems and methods described herein relate to the field of endoscopic instruments. More specifically, the devices, systems and methods described herein utilize overtube devices with novel features for use with endoscopic instruments that allow for more diagnostic and therapeutic interventions than previously possible.

### BACKGROUND OF THE INVENTION

Less invasive surgical procedures can reduce patient trauma and, as a result, may reduce the length of hospital stays, as well as hospital and medical costs. Endoscopic surgery recently has provided a significant opportunity to reduce the invasiveness of numerous surgical procedures. This type of surgery involves the use of an endoscope, an instrument that permits the visual inspection and magnification of cavities within the body. An endoscope may be inserted through a small surgical incision to view organ structures in a body cavity or through a natural orifice such as the mouth, anus, bladder, and vagina to view channel-containing organs in the gastrointestinal, respiratory, and genital and urinary tracts. Endoscopes generally have channels along their length to introduce instrumentation for functions such as irrigation or suction, and for the insertion of accessory instruments when a surgical procedure is performed. While endoscopes provide a number of advantages, presently available diagnostic and therapeutic interventions are restricted by the dimension of an endoscope's working channel that permit only a limited number of instruments to be advanced to a treatment site at a time. While some endoscope accessory devices provide sheaths with additional channels for instrumentation, these devices have certain restrictions (such as a requirement to be placed around the endoscope prior to insertion into a patient) and do not allow for control over a variety of additional tools that may be found at the end of an endoscope. Thus, an advance that allowed for additional diagnostic and therapeutic interventions through the use of endoscopes by allowing the introduction of more instruments at a treatment site, without the restrictions associated with previous devices, would provide a great benefit. The present invention provides such an advance.

### SUMMARY OF THE INVENTION

The present invention provides devices, systems and methods to increase the potential number of diagnostic and therapeutic interventions performed through endoscopes without the restrictions associated with previous devices. The present invention increases the potential for diagnostic and therapeutic interventions by providing access channels outside the normal working channel of an endoscope that, in one embodiment, can be introduced after the endoscope has been inserted into a patient. These access channels are provided in devices presently called overtubes that fit around a conventional endoscope. The overtubes of the present invention can include one or more additional channels through which instrumentation can be deployed, increasing the number of tools that can be concurrently used at a treatment site with the deployment of one endoscope. Further, overtubes of the present invention can be used in conjunction with devices that are pre-placed around an endoscope (“pre-placed devices”). Pre-placed devices can be used to perform a number of functions that enhance diagnostic and therapeutic objectives outside the conventional channel of an endoscope. Some of these functions can include, without limitation, providing a space to adjust the distance of the endoscope tip to tissue, detecting changes in tissue (including, without limitation macro- and or microstructural, biochemical or molecular changes), providing a space to aspirate and hold tissue, providing a medium to enhance optics, deploying a stent, deploying a ligating element (including, without limitation a band or a loop), deploying a cutting instrument (including, without limitation, a snare or a needle knife) with diathermy, deploying a cutting instrument without diathermy, deploying an anchoring or joining element (including, without limitation, a stitch, staple, or T-tag), and deploying tissue ablative energy (including, without limitation, thermal, photochemical, laser, microwave and radiofrequency).

Specifically, one embodiment according to the present invention is a device comprising a flexible tube and one or more open channels within the wall of the flexible tube wherein the tube further comprises a break along its length and the inner circumference of the tube is within about 0.5 mm of the outer perimeter of an endoscope and wherein the flexible tube is introduced onto the endoscope with the use of an introducer.

In another embodiment of the device, the one or more open channels within the wall of the flexible tube extend the length of the tube. In another embodiment of the device, the lengthwise break can be closed. In another embodiment of the device, the one or more open channels are collapsed in their free state. In another embodiment of the device, the flexible tube is segmented along its length with sections of semi-rigid and flexible materials. In another embodiment of the device, the sections of semi-rigid and flexible materials alternate along the length of the flexible tube. In another embodiment of the device, the device further comprises a clamp mechanism at or near the proximal end of the flexible tube. In another embodiment of the device, the one or more of the open channels house a control wire that terminates proximally in a control device. In another embodiment of the device, the distal end of the control wire comprises one or more hooks. In another embodiment of the device, one end and a portion of the flexible tube adjacent thereto have a perimeter that is reduced compared to the rest of the flexible tube. In another embodiment of the device, the reduced perimeter portion of the flexible tube comprises one or more lugs.

Another embodiment of the present invention comprises an endoscope with an introducer for use with one of the devices of the present invention.

The present invention also comprises systems. In one embodiment of the systems according to the present invention, the system comprises a first device comprising a flexible tube and one or more open channels within the wall of the flexible tube wherein the tube further comprises a break along its length and the inner circumference of the tube is within about 0.5 mm of the outer perimeter of an endoscope and a second device wherein the second device comprises a tube with an inner circumference of within about 0.5 mm of the outer perimeter of the endoscope.

In another embodiment of the systems, the one or more open channels within the wall of the first device extend the length of the first device. In another embodiment of the systems, the lengthwise break of the first device can be closed. In another embodiment of the systems, the one or more open channels within the wall of the first device are collapsed in their free state. In another embodiment of the systems, the flexible tube of the first device is segmented along its length with sections of semi-rigid and flexible materials. In another embodiment of the systems, the sections of semi-rigid and flexible materials alternate along the length of the flexible tube of the first device. In another embodiment of the systems, the first device further comprises a clamp mechanism at or near its proximal end. In another embodiment of the systems, the one or more of the open channels within the wall of the first device house a control wire that terminates proximally in a control device. In another embodiment of the systems, the distal end of the control wire comprises one or more hooks. In another embodiment of the systems, the first device and the second device can be linked. In another embodiment of the systems, on the first device, one end and a portion of the first device adjacent thereto have a perimeter that is reduced compared to the rest of the first device. In another embodiment of the systems, the reduced perimeter portion of the first device fits within the perimeter of the second device. In another embodiment of the systems, the reduced perimeter portion of the first device further comprises one or more lugs and one end and a portion adjacent thereto of the second device comprise one or more slots configured to receive the one or more lugs. In another embodiment of the systems, the one or more lugs and the one or more slots can securely link the first device and the second device. In another embodiment of the systems, the link is in the form of a bayonet joint. In another embodiment of the systems, the second device comprises an actuator member. In another embodiment of the systems, the second device comprises an actuator that comprises an eyelet and wherein when the first device and the second device are linked, the hook of the wire engages the eyelet of the actuator member and the wire can be used to control the second device. In another embodiment of the systems, the second device is adapted to perform a function selected from the group consisting of providing a space to adjust the distance of the endoscope tip to tissue, detecting changes in tissue (including, without limitation macro- and or microstructural, biochemical or molecular changes), providing a space to aspirate and hold tissue, providing a medium to enhance optics, deploying a stent, deploying a ligating element (including, without limitation a band or a loop), deploying a cutting instrument (including, without limitation, a snare or a needle knife) with diathermy, deploying a cutting instrument without diathermy, deploying an anchoring or joining element (including, without limitation, a stitch, staple, or T-tag), and deploying tissue ablative energy (including, without limitation, thermal, photochemical, laser, microwave and radiofrequency).

The present invention also includes an endoscope comprising an introducer for use with the systems of the present invention.

In one embodiment according to the present invention, the invention further comprises a device comprising a tubular portion with an inner perimeter of within about 0.5 mm of the outer perimeter of an endoscope wherein the device comprises an actuator. In another embodiment of the device, the actuator comprises an eyelet. In another embodiment of the device, the device is adapted to perform a function near the end of an endoscope selected from the group consisting of providing a space to adjust the distance of the endoscope tip to tissue, detecting changes in tissue (including, without limitation macro- and or microstructural, biochemical or molecular changes), providing a space to aspirate and hold tissue, providing a medium to enhance optics, deploying a stent, deploying a ligating element (including, without limitation a band or a loop), deploying a cutting instrument (including, without limitation, a snare or a needle knife) with diathermy, deploying a cutting instrument without diathermy, deploying an anchoring or joining element (including, without limitation, a stitch, staple, or T-tag), and deploying tissue ablative energy (including, without limitation, thermal, photochemical, laser, microwave and radiofrequency).

The present invention also comprises methods. In one method according to the present invention, the method comprises performing an endoscopic procedure on a patient with an endoscope, wherein the endoscope is associated with an overtube, wherein the overtube comprises a flexible tube and one or more open channels within the wall of the flexible tube wherein the tube further comprises a break along its length and the inner circumference of the tube is within about 0.5 mm of the outer perimeter of the endoscope and wherein the overtube is introduced onto the endoscope with the use of an introducer.

In another embodiment of the methods, the one or more open channels within the wall of the flexible tube extend the length of the tube. In another embodiment of the methods, the lengthwise break can be closed. In another embodiment of the methods, the endoscope comprises an introducer. In another embodiment of the methods, the one or more open channels are collapsed in their free state. In another embodiment of the methods, the overtube is segmented along its length with sections of semi-rigid and flexible materials. In another embodiment of the methods, the semi-rigid and flexible materials alternate along the length of the overtube. In another embodiment of the methods, the overtube further comprises a clamp mechanism at or near its proximal end. In another embodiment of the methods, the one or more of the open channels house a control wire that terminates proximally in a control device operated by a person involved in the performing of the endoscopic procedure. In another embodiment of the methods, the distal end of the control wire comprises one or more hooks. In another embodiment of the methods, one end and a portion of the overtube adjacent thereto have a perimeter that is reduced compared to the rest of the overtube. In another embodiment of the methods, the reduced perimeter portion of the overtube comprises one or more lugs.

In another embodiment of the methods, the performing of the endoscopic procedure further comprises associating a pre-placed device with the endoscope wherein the pre-placed device comprises a tube with an inner circumference of within about 0.5 mm of the outer perimeter of the endoscope and the associating occurs through placing the pre-placed device around the outer perimeter of the endoscope. In another embodiment of the methods, the performing of the endoscopic procedure further comprises linking the overtube and the pre-placed device. In another embodiment of the methods, the one or more of the open channels house a control wire that terminates proximally in a control device operated by a person involved in the performing of the endoscopic procedure. In another embodiment of the methods, the distal end of the control wire comprises one or more hooks. In another embodiment of the methods, the linking occurs through the use of on or more lugs and one or more slots to form a bayonet joint. In another embodiment of the methods, the pre-placed device comprises an actuator member. In another embodiment of the methods, the pre-placed device comprises an actuator that comprises an eyelet and wherein when the overtube and the pre-placed device are linked, the hook of the wire engages the eyelet of the actuator member and the wire can be used to control the pre-placed device. In another embodiment of the methods, one aspect of the endoscopic procedure comprises using the pre-placed device to perform a function selected from the group consisting of providing a space to adjust the distance of the endoscope tip to tissue, detecting changes in tissue (including, without limitation macro- and or microstructural, biochemical or molecular changes), providing a space to aspirate and hold tissue, providing a medium to enhance optics, deploying a stent, deploying a ligating element (including, without limitation a band or a loop), deploying a cutting instrument (including, without limitation, a snare or a needle knife) with diathermy, deploying a cutting instrument without diathermy, deploying an anchoring or joining element (including, without limitation, a stitch, staple, or T-tag), and deploying tissue ablative energy (including, without limitation, thermal, photochemical, laser, microwave and radiofrequency).

Another method according to the present invention comprises assembling an endoscopic device wherein the endoscopic device comprises an endoscope and an overtube, wherein the overtube comprises a flexible tube and one or more open channels within the wall of the flexible tube wherein the tube further comprises a break along its length and the inner circumference of the tube is within about 0.5 mm of the outer perimeter of the endoscope and wherein the assembling comprises placing the overtube around the outer perimeter of the endoscope with the use of an introducer.

In another assembling method, the one or more open channels within the wall of the flexible tube extend the length of the tube. In another assembling method, the lengthwise break can be closed. In another assembling method, the endoscope comprises an introducer for use in the placing of the overtube. In another assembling method, the one or more open channels are collapsed in their free state. In another assembling method, the flexible tube is segmented along its length with sections of semi-rigid and flexible materials. In another assembling method, the sections of semi-rigid and flexible materials alternate along the length of the flexible tube. In another assembling method, the overtube further comprises a clamp mechanism at or near its proximal end and the method further comprises clamping the mechanism after the placing. In another assembling method, the one or more of the open channels house a control wire that terminates proximally in a control device. In another assembling method, the distal end of the control wire comprises one or more hooks.

In another assembling method, the assembling further comprises associating the endoscope of the endoscopic device with a pre-placed device, the pre-placed device comprising a tube with an inner circumference of within about 0.5 mm of the outer perimeter of the endoscope and wherein the associating comprises placing the pre-placed device around the outer perimeter of the endoscope. In another assembling method, the method further comprises linking the overtube and the pre-placed device. In another assembling method, the one or more of the open channels house a control wire that terminates proximally in a control device. In another assembling method, the distal end of the control wire comprises one or more hooks. In another assembling method, the linking occurs through the use of a bayonet joint using one or more lugs on the overtube and one or more slots on the pre-placed device. In another assembling method, the pre-placed device comprises an actuator member. In another assembling method, the pre-placed device comprises an actuator that comprises an eyelet and wherein when the overtube and the pre-placed device are linked, the hook of the wire engages the eyelet of the actuator member and the wire can be used to control the pre-placed device. In another assembling method, the pre-placed device is adapted to perform a function selected from the group consisting of providing a space to adjust the distance of the endoscope tip to tissue, detecting changes in tissue (including, without limitation macro- and or microstructural, biochemical or molecular changes), providing a space to aspirate and hold tissue, providing a medium to enhance optics, deploying a stent, deploying a ligating element (including, without limitation a band or a loop), deploying a cutting instrument (including, without limitation, a snare or a needle knife) with diathermy, deploying a cutting instrument without diathermy, deploying an anchoring or joining element (including, without limitation, a stitch, staple, or T-tag), and deploying tissue ablative energy (including, without limitation, thermal, photochemical, laser, microwave and radiofrequency).

### BRIEF DESCRIPTION OF THE DRAWINGS

FIG. 1 is a perspective view of a typical flexible endoscope known in the art;

FIG. 2 is a perspective view of one embodiment of an overtube of the present invention in position to be installed around a flexible endoscope;

FIG. 3 is a perspective view of an overtube according to the present invention in its installed position around a flexible endoscope;

FIG. 4 is a cross-sectional view of one embodiment of an overtube according to the present invention;

FIG. 5 is a cross-sectional view of an alternative embodiment of an overtube;

FIG. 6 is a cross-sectional view of the overtube depicted in FIG. 5 with the open channels expanded by the insertion of devices;

FIG. 7 is a cross-sectional view of an overtube with a zip-lock type joint;

FIG. 8 is a detail of the zip-lock type joint depicted in FIG. 7;

FIG. 9 is a perspective view of the distal end of an overtube with filler sections removed for illustrative purposes;

FIG. 10 is a perspective view, similar to FIG. 9, showing the complete overtube with filler sections in place;

FIG. 11 is a perspective view of an introducer used to ease the installation of an overtube onto the shaft of a flexible endoscope;

FIGS. 12 and 13 show perspective views of the proximal end of an overtube with a clamping mechanism;

FIG. 14 shows, in a series of three views (14a, 14b and 14c, respectively), the sequence of steps in connecting a bayonet joint between an overtube and a device pre-placed onto an endoscope shaft;

FIG. 15a shows a detailed perspective view of the distal end of an overtube with provision for a bayonet joint;

FIG. 15b shows an end view of the overtube depicted in FIG. 15a;

FIG. 16 depicts a cross-sectional view of the overtube shown in FIG. 15b;

FIG. 17a is an end view of the assembly of the pre-placed device and overtube depicted in FIG. 14c;

FIG. 17b is a cross-sectional view of a portion of the assembly depicted in FIGS. 14c and 17a detailing the connection of control wires and actuator members between the pre-placed device and the overtube;

FIG. 18, consisting of FIGS. 18a and 18b, shows an embodiment of a pre-placed cap device connected to an overtube with a bayonet joint;

FIG. 19 shows, in a series of three views (FIGS. 19a, 19b and 19c, respectively), a sequence of steps for using a pre-placed band ligating device; and

FIG. 20 shows, in a series of three views (FIGS. 20a, 20b and 20c, respectively), a sequence of steps for using a pre-placed stent delivery device.

### DETAILED DESCRIPTION OF THE INVENTION

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

In the following description it should be noted that directional terms such as “distal” and “proximal” are used relative to each other and do not refer to positions or orientations relative to an external frame of reference. Also, “locked” and “interlocked” are coextensive in meaning.

The terms “endoscope” or “endoscopic” refer not only to conventional endoscopes and endoscopic procedures, but also to any rigid, semi-rigid, or flexible optical instrument for use in visual examinations where access is limited to a small incision or opening. Generally, such examinations will occur in the human body, however, the use of the terms is not so restricted. References to endoscopes and endoscopic procedures are understood also to encompass procedures in all organisms, living or dead, as well as, the examination of inanimate objects through small openings. Endoscopes and endoscopic procedures are understood to include laparoscopic devices and laproscopic procedures. The term “endoscope” also includes echo-endoscopes, which may include an ultrasound transducer at, for example, the tip of the device.

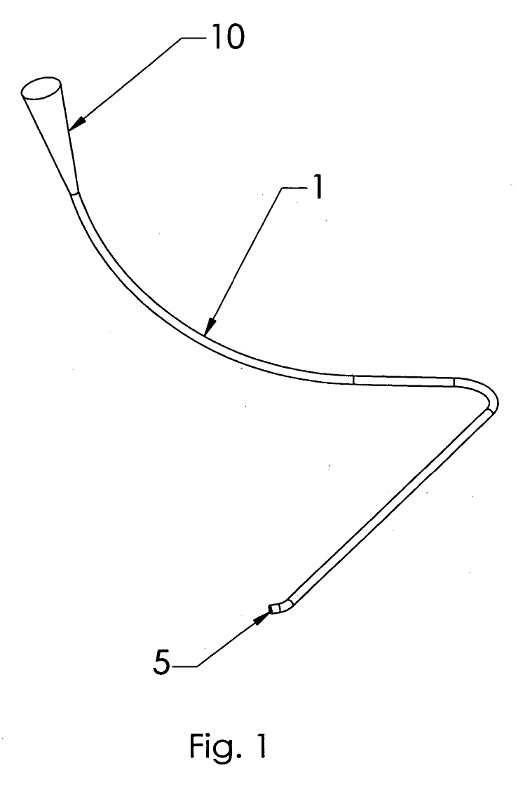

FIG. 1 through FIG. 20 depict exemplary devices, systems and methods of the present invention. These devices, systems and methods are depicted and described herein in order to better explain the invention. It will be understood that the devices, systems and methods shown are representative only, and that devices of other configurations, sizes and styles are within the scope of the present invention.

FIG. 1 is a perspective view of a flexible endoscope 1 as known in the prior art. The endoscope has a distal end 5 that is inserted into a body cavity of a human or animal. The distal end of the endoscope is equipped with means for illuminating and viewing inside the body cavity. The endoscope is typically fitted with a channel for the insertion of various tools into the body. The end of this channel is also located at the distal end of the endoscope. The endoscope has a proximal end 10 that includes controls for flexing the distal end of the endoscope among others controlling function.

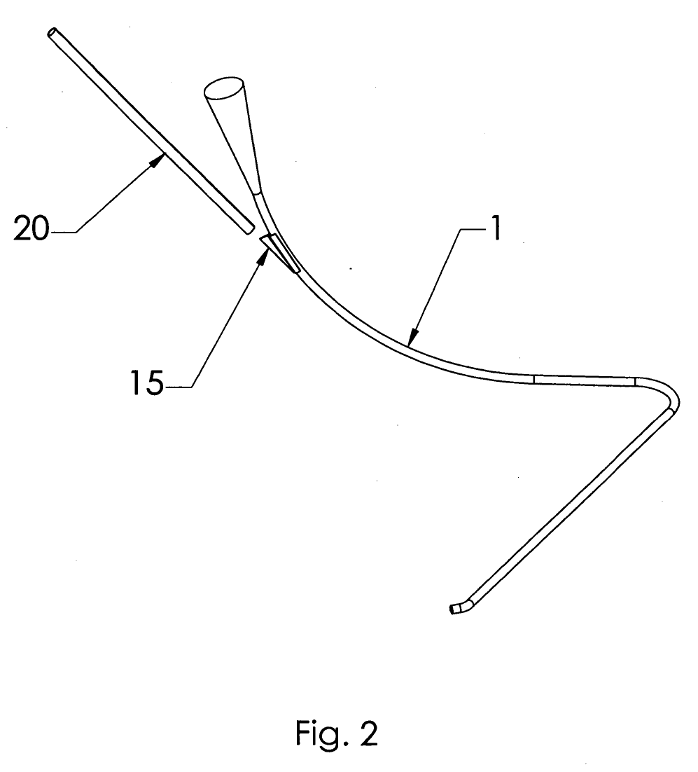

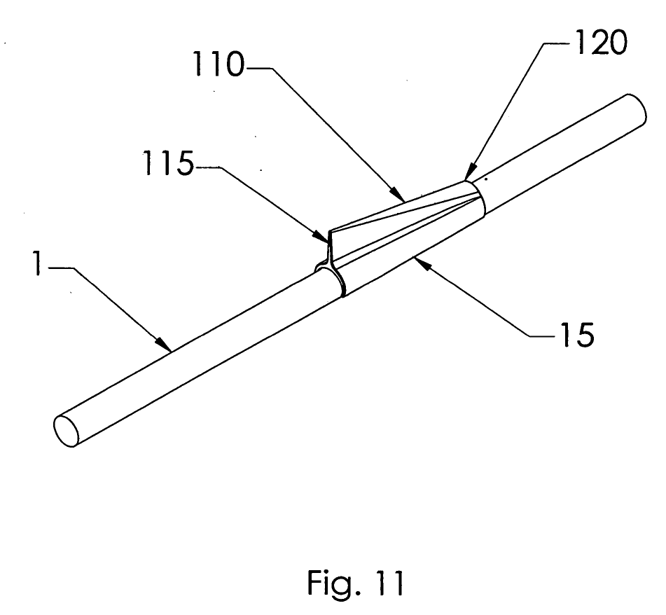

FIG. 2 is a perspective view of a flexible endoscope 1 and a section of an overtube 20 in accordance with one embodiment of the present invention. The section of overtube 20 is shown in position ready to be installed around the endoscope. In this embodiment, an introducer 15 is attached to the endoscope in order to aid in the installation of the overtube 20. This feature will be described in more detail in relation to FIG. 11. In one embodiment of the present invention, the overtube 20 is installed around the endoscope after the endoscope has been placed into the body cavity at an approximate area of interest.

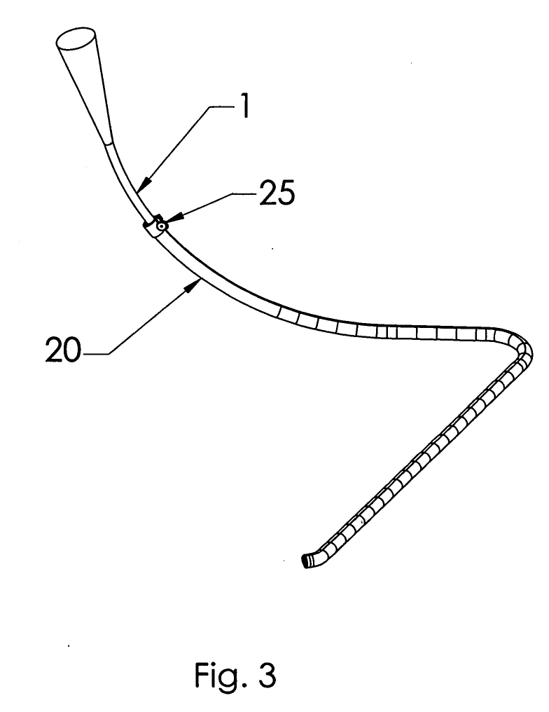

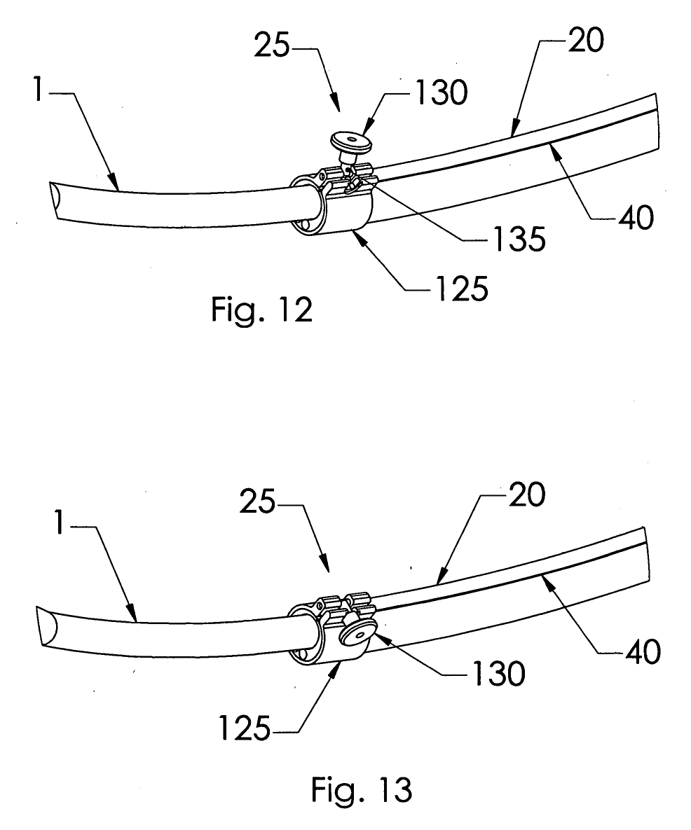

FIG. 3 is a perspective view of a flexible endoscope 1 with an overtube 20 fully attached around the endoscope 1. In the depicted embodiment, a clamp 25 serves to hold the overtube 20 in position relative to the endoscope 1. Operation of the clamp will be described in more detail in relation to FIGS. 12 and 13.

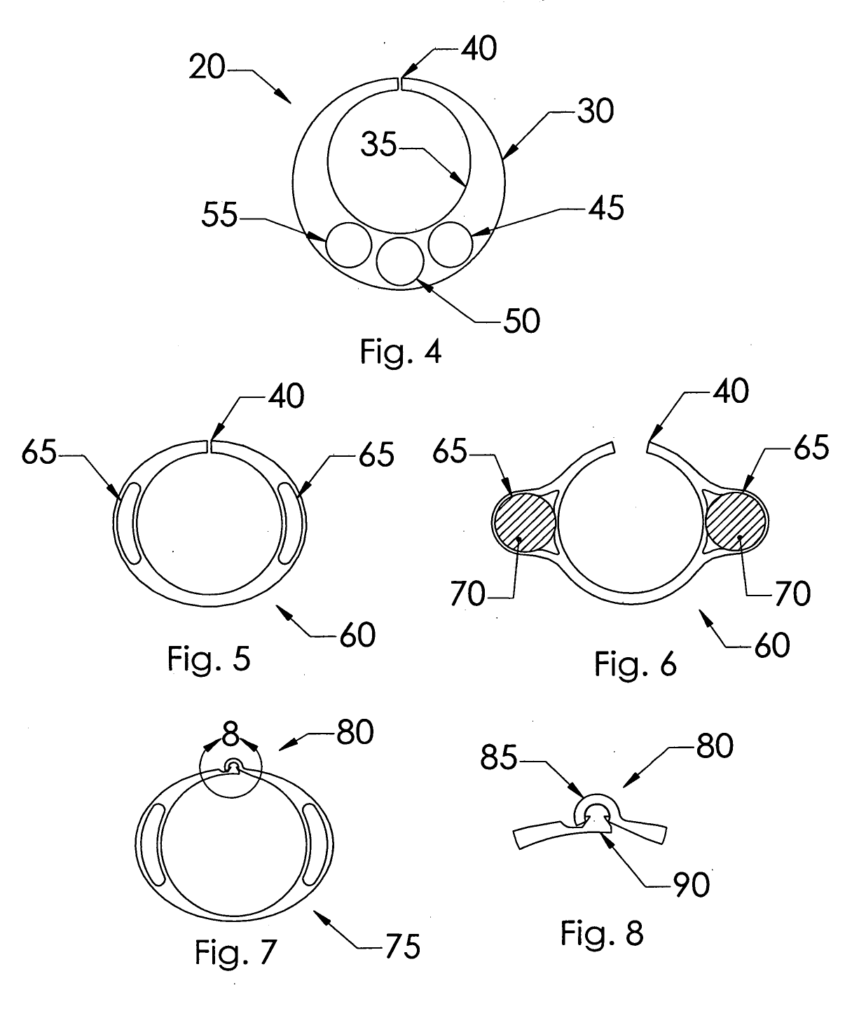

FIG. 4 depicts a cross-section taken normal to the long axis of an overtube 20. The wall 30 of the overtube 20 encompasses channels 45, 50 and 55. The number and size of channels included in a particular overtube are variable and can be a function of the overtube's intended usage and can include one or more, two or more, three or more, four or more, or five or more channels. These channels add extra functionality to the flexible endoscope by allowing the delivery of additional instrumentation to an area of interest. The ability to deliver additional instrumentation with the insertion of one endoscope can increase the number of possible interventions. The inner surface 35 of the overtube can be sized to provide a close fit to the endoscope in use, such that it is large enough not to bind the endoscope but small enough to be effectively guided by the endoscope. In a specific embodiment, the depicted break 40 in the overtube wall 30 allows the overtube 20 to be installed over the proximal end of an endoscope while the distal end of the endoscope is within a body cavity. Generally, the break 40 may run the entire length of the overtube 20. In practice, the overtube 20 can be elastically deformed by the bending of its wall 30 to open the break 40 to a size that allows the endoscope to pass inside the overtube 20. This elastic deformation may extend over only a relatively small axial length of the overtube 20, and progresses over the length of the overtube 20 as it is installed around an endoscope. This deformation can be aided by an introducer 15, again to be described in more detail in relation to FIG. 11.

FIG. 5 depicts a cross-section taken normal to the long axis of another embodiment of an overtube 60 according to the present invention. This overtube 60 has two channels 65 that are “collapsed” or highly elongated in their free state. In this embodiment with collapsed channels, the outside perimeter of the overtube 60 is reduced compared to the outside perimeter of the overtube 20 depicted in FIG. 4. This smaller outside perimeter can aid in the insertion of an overtube into a body cavity by reducing the magnitude of the “step-off” or distance between the perimeter of the endoscope shaft and the perimeter of the overtube. The channels 65 in the overtube 60 can be made of a sufficiently flexible material to allow them to deform when a device or instrumentation is passed through them. The overtube 60 depicted in FIG. 5 has a break 40 that is similar to the break 40 depicted in FIG. 4.

FIG. 6 depicts a cross-section taken normal to the long axis of the same embodiment of the overtube 60 as shown in FIG. 5. The channels 65 in this FIG. 6, however, have been expanded by the passage of a device 70 or instrumentation. As depicted, the expansion of the channels 65 can cause a deformation of the overtube 60 that increases the separation of the break 40.

FIG. 7 depicts a cross-section taken normal to the long axis of another embodiment of an overtube 75. In this depicted embodiment instead of a break, the embodiment has a joint 80 that can be opened and closed (in one embodiment resealed) multiple times. The joint can be similar to those found on resealable plastic bags commonly referred to as zip-lock seals. FIG. 8 shows a detail view of one embodiment that comprises a resealable joint 80. The depicted joint 80 is made up of two members: the upper member 85 and the lower member 90. The depicted lower member 90 has a cross-section in a mushroom profile. The depicted upper member 85 has a circular profile that can snap over the mushroom profile. The sealed nature of this joint can be preferable for some overtube applications for instance when the space between an overtube and an endoscope must maintain a vacuum or set pressure.

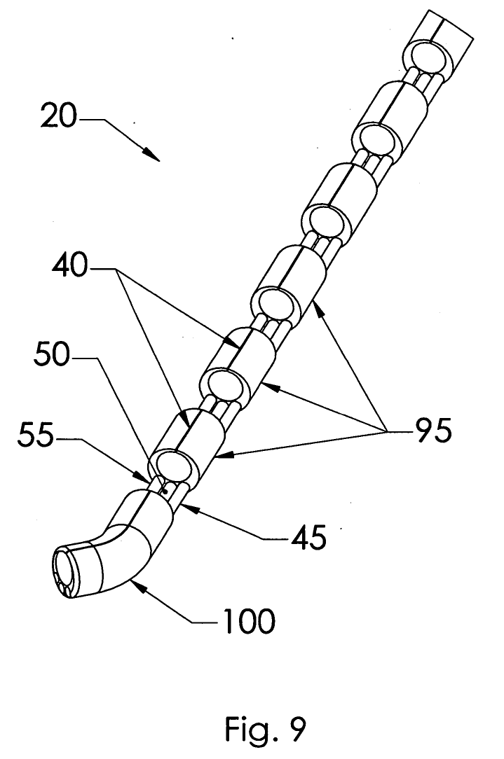

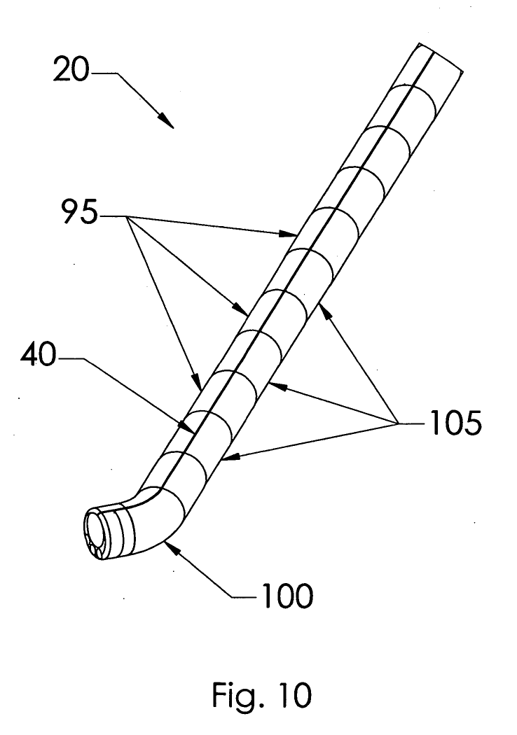

FIG. 9 is a perspective view of the distal end of a segmented overtube 20 with filler portions between segments removed for illustrative purposes. Overtubes of the present invention can be segmented to provide flexibility so that the overtube can more easily follow a curved path taken by a flexible endoscope. In one embodiment, segmentation can be accomplished by alternating sections of a semi-rigid material 95, with flexible filler sections 105 (see FIG. 10). The channels 45, 50 and 55 of an overtube can be continuous and can run the full length of the overtube 20. The break 40 also can run the full length of the overtube and can be present in each semi-rigid section 95. To accommodate the controlled bending at the tip of an endoscope, a highly flexible section 100 of the overtube 20 can be present at its distal end. This flexible tip section 100 can also have a break 40. In one embodiment the semi-rigid sections 95 can be preferably molded or extruded from without limitation, a plastic material such as polypropylene, polyurethane, nylon, polyethylene terephthalate, polytetrafluoroethylene (PTFE), or silicone, a metal material such as nitinol or stainless steel, or a plastic material reinforced with a metal material.

The channels can be extruded from a plastic material, preferably with good strength and a low coefficient of friction, and in one embodiment may be coated with Teflon® for superior frictional properties. The flexible tip section 100 can be molded from an elastomeric material such as, without limitation, silicone. As will be understood by one of ordinary skill in the art, a number of other materials can be appropriate for use in the flexible portions of the presently disclosed devices.

FIG. 10 is the same perspective view as in FIG. 9 but with the flexible filler sections 105 present. In one embodiment the flexible filler sections 105 can be bonded or attached to the semi-rigid sections 95 and flexible tip section 100 to form a unitary structure. If the sections 95, 100 and 105 are unitized as an assembly, the break 40 can be made through the flexible tip section 100 and all the flexible filler sections 105 at once and in line with the breaks 40 in individual semi-rigid sections 95.

FIG. 11 is a perspective view of an introducer 15 positioned on a flexible endoscope 1 to ease the process of installing an overtube onto the endoscope. The blade 110 on the depicted introducer has a thin end 115 and a thick end 120. The thin end 110 can have a thickness about equal to or slightly less than the width of the break 40 in an overtube. The overtube can be easily slipped onto the thin end 115 of the introducer blade 110. The overtube can then be advanced along the length of the introducer and pressed towards the endoscope shaft. As the introducer blade 110 gradually widens towards its thick end 120 the overtube is elastically deformed and the break 40 is opened to a dimension that allows the overtube to accept the endoscope within the inner perimeter of its wall. While the introducer described provides a beneficial feature, they are not required as it is anticipated that the overtube could also be manually placed or snapped onto an endoscope without the aid of an introducer.

FIGS. 12 and 13 provide perspective views of one embodiment of a clamp mechanism 25 that can secure an overtube 20 to an endoscope 1 so that their relative positions remain stable during use. The clamp mechanism may comprise a clamp body 125 that can be integral with the proximal portion or end of an overtube 20. A pivoting tee-bolt 135 can be constrained within the clamp body 125. A clamp nut 130 can engage with the threads of the pivoting tee-bolt 135. As shown in FIG. 12, the clamp nut 130 can be threaded out and the pivoting tee bolt 135 can be free to move. The overtube could be installed in this configuration so the break 40 is free to open and snap over the endoscope 1. When it is desired to fix the relative positions of the overtube and the endoscope, in one embodiment the clamp 25 can be locked as shown in FIG. 13. To lock the clamp, the pivoting tee-bolt 135 can be rotated over and the clamp nut 130 can be tightened so that the clamp body 125 squeezes the overtube and endoscope and the resultant frictional force holds the overtube in place.

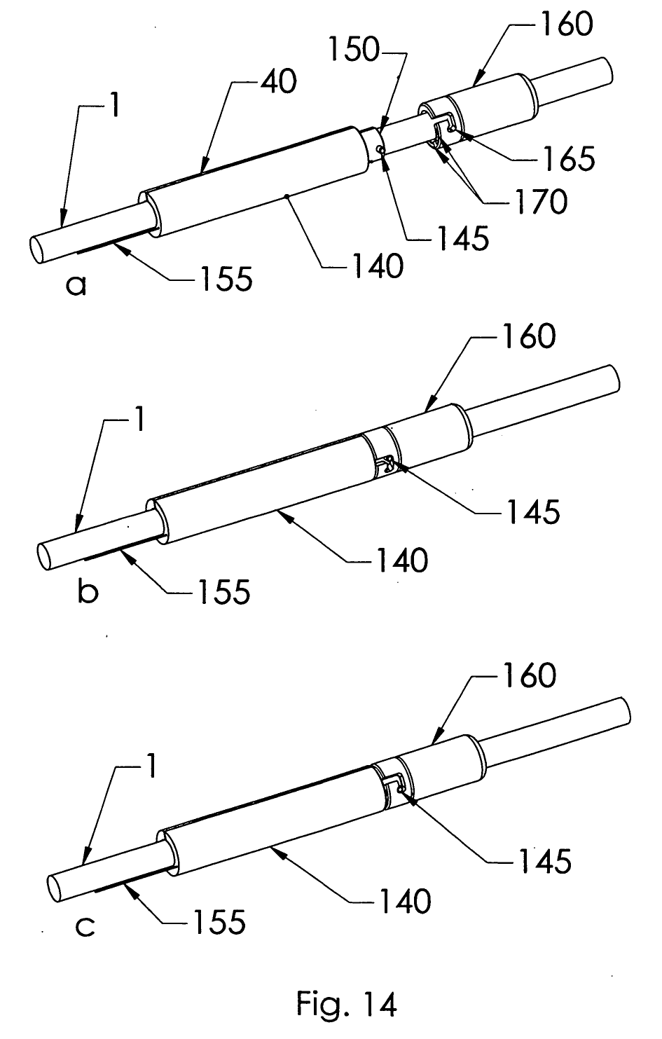

FIG. 14 provides three perspective views of the assembly sequence of a pre-placed device 160 with a further embodiment of an overtube 140 of the present invention. A pre-placed device is a device that is placed over an endoscope prior to the start of a procedure. In practice, the pre-placed device generally may be fixed near the proximal end of an endoscope where it will initially not enter the body cavity. Pre-placed devices can be used with overtubes of the present invention to achieve a variety of functions including, without limitation, providing a space to adjust the distance of the endoscope tip to tissue, detecting changes in tissue (including, without limitation macro- and or microstructural, biochemical or molecular changes), providing a space to aspirate and hold tissue, providing a medium to enhance optics, deploying a stent, deploying a ligating element (including, without limitation a band or a loop), deploying a cutting instrument (including, without limitation, a snare or a needle knife) with diathermy (the use of electrical current as a cutting device or to stop bleeding during surgery), deploying a cutting instrument without diathermy, deploying an anchoring or joining element (including, without limitation, a stitch, staple, or T-tag), and deploying tissue ablative energy (including, without limitation, thermal, photochemical, laser, microwave and radiofrequency). It should be understood that a number of devices and procedures can be adapted for use as (or with) pre-placed devices of the present invention. Non-limiting examples include tools and methods similar to those shown in U.S. Pat. No. 6,953,430 (pincer like instruments for use with an endoscope); U.S. Pat. No. 6,966,906 (deflection instrument for a surgical instrument used with an endoscope); U.S. Pat. No. 6,206,904 (foreign body recovery device); U.S. Pat. No. 5,683,413 (forceps for use with an endoscope); U.S. Pat. Nos. 6,258,083; 6,174,307; 6,902,526; and U.S. Patent Application No. (USPAN) 20050182298 (tissue ablation procedures); USPAN 20060058703 (optical biopsy instrument); USPAN 20060047279 (polypectomy snare); and USPAN 20060030756 (vein harvesting tools). As will be understood by one of ordinary skill in the art, these functions, devices and methods can be performed alone or in appropriate combinations, such as, without limitation, deploying a band or snare after aspirating and holding tissue.

In one embodiment, the pre-placed device can be protected with a covering that can maintain the cleanliness or sterility of the pre-placed device. The coverings according to the present invention can adopt many different forms including, without limitation, easily removable bag-type coverings, protective foam coverings or rigid or semi-rigid plastic coverings. Generally, an endoscopist will select and install a pre-placed device around an endoscope based on the type of intervention he/she expects to perform during a procedure. If the particular intervention is not required, the pre-placed device can be removed from the endoscope after the procedure and may be reused at a later time.

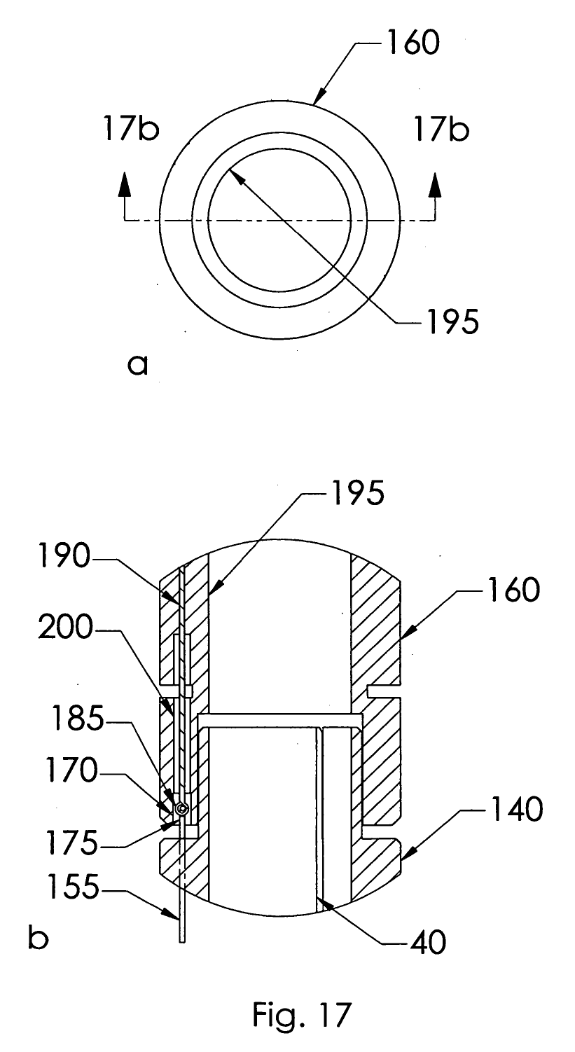

When the endoscopist determines that he/she will use a pre-placed device during a procedure, an overtube 140 can be partially installed on the endoscope as described earlier. FIG. 14a illustrates a section of overtube 140 on the endoscope 1 near a pre-placed device 160. Pre-placed devices used in accordance with the present invention may require actuation by the endoscopist to perform its intended function. Provision for actuation of a pre-placed device can be made with a control wire 155 that passes through a channel that runs the full length of the overtube 140 and terminates in a control device manipulated by the endoscopist. The overtube 140 has a break 40 running its full length as previously described to allow installation of the overtube on the endoscope. In one embodiment, the overtube and pre-placed device can be connected by way of a bayonet joint. In this embodiment, the overtube can have a reduced perimeter portion 150 with one or more lugs 145. The lugs 145 can engage slots 165 in the pre-placed device 160. Thus, a push and twist action can connect the overtube and pre-placed device securely. Visible in FIG. 14a are eyelet recesses 170 that will be described more completely with regard to FIG. 17. FIG. 14b shows the bayonet joint partially made: the pre-placed device 160 and the overtube 140 have been pushed together. In FIG. 14c, the pre-placed device 160 has been twisted relative to the overtube 140 and the bayonet joint has been secured.

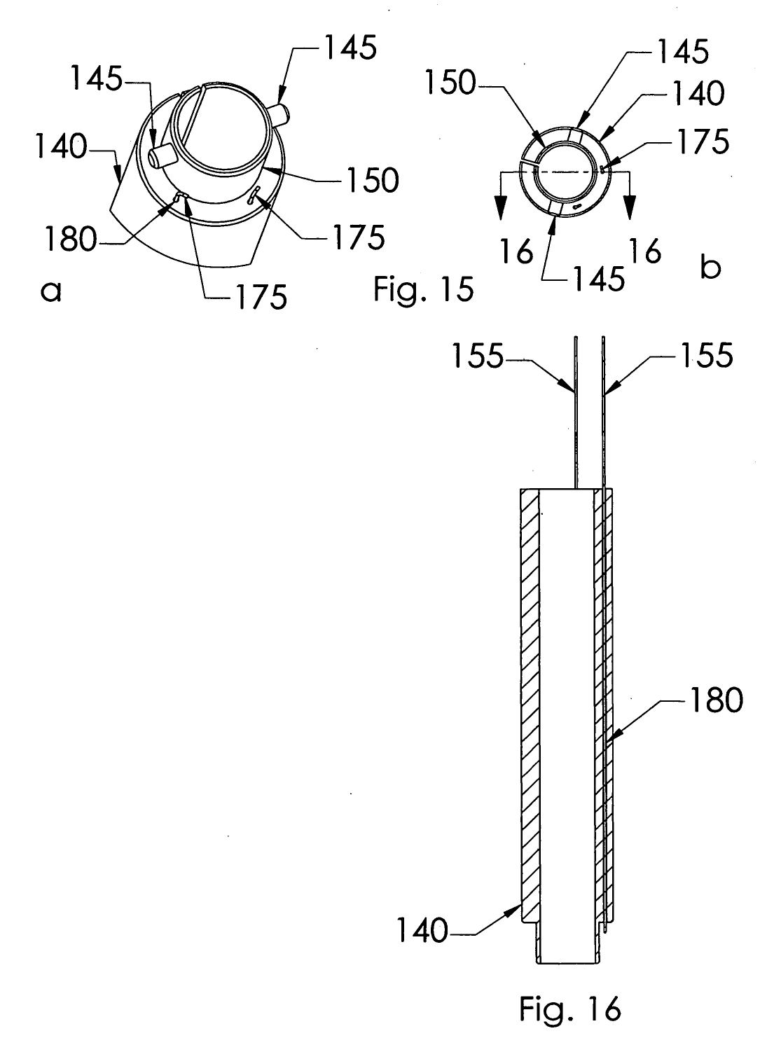

FIG. 15a is a perspective view detail of the distal end of an overtube 140. Bayonet lugs 145 are visible protruding from a reduced perimeter portion 150. The distal end of the control wires 155 are provided with hooks 175. In this figure two control wires are illustrated; in practice any number of control wires from zero to as many as three or four or more could be implemented. The control wire 155 can run through the channel 180 in the overtube 140.

FIG. 15b is an end view of the distal end of the overtube 140. Section 16-16 is taken through this view and is shown in FIG. 16. The control wire 155 and its channel 180 in the overtube 140 are shown in FIG. 16.

FIG. 17a depicts an end view of an assembly of pre-placed device 160 and overtube 140 as shown in FIG. 14c. The inside surface 195 of the pre-placed device 160 is shown in the drawing. Section 17b-17b is taken through the view in FIG. 17a and is shown in FIG. 17b. FIG. 17b illustrates how the control wires 155 in overtube 140 can be connected to actuator members 190 in pre-placed device 160. An eyelet 185 integral with the actuator member 190 can be engaged by the hook end 175 of control wire 155 when the pre-placed device 160 and overtube 140 are pushed together and twisted relative to each other to form a bayonet joint. The eyelet recess 170 (also visible in FIG. 14a) allows room for a protruding hook end 175 of control wire 155 to sweep through an arc as the pre-placed device 160 and overtube 140 are twisted together. Also shown in FIG. 17b is a channel 200 in the pre-placed device for actuator member 190. Break 40 and inside surface 195 of the pre-placed device are also indicated. The engagement of the hook end 175 of the control wire 155 with the eyelet 185 allows transmission of tension and compression loads through the control wire 155 to the actuator member 190 in a pre-placed device. Uni-directional twisting action of the control wire 155 may also be transmitted through the junction of the hook end 175 with the eyelet 185.

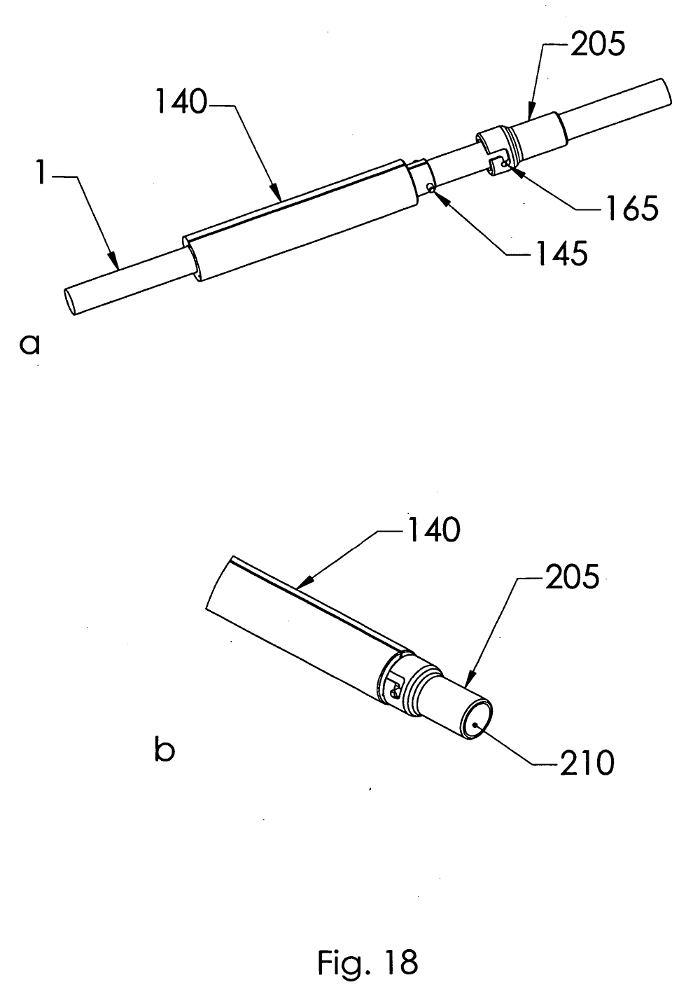

FIG. 18 illustrates an embodiment of a pre-placed device with a bayonet joint. In the depicted embodiment, the pre-placed device is a cap intended to act as either a spacer to either maintain an appropriate distance of the endoscope tip from tissue, enable the focus of endoscope optical elements, or act as a spacer for aspiration and retention of tissue to be resected, ligated, stitched, stapled or ablated. FIG. 18a is a perspective view illustrating the pre-placed cap device 205 mounted on an endoscope 1. A section of overtube 140 with provision for a bayonet joint connection is shown in position on the endoscope 1. Bayonet lugs 145 on the overtube 140 are aligned with bayonet slots 165 on the pre-placed cap 205. The pre-placed cap 205 and overtube 140 are then pushed together and twisted relative to each other to make the bayonet connection. FIG. 18b is a detail perspective view of the pre-placed cap 205 and overtube 140 once connected. The inner volume 210 of the pre-placed cap 205 is visible in this drawing. By relative motion of the endoscope and the overtube, the size of the inner volume may be adjusted.

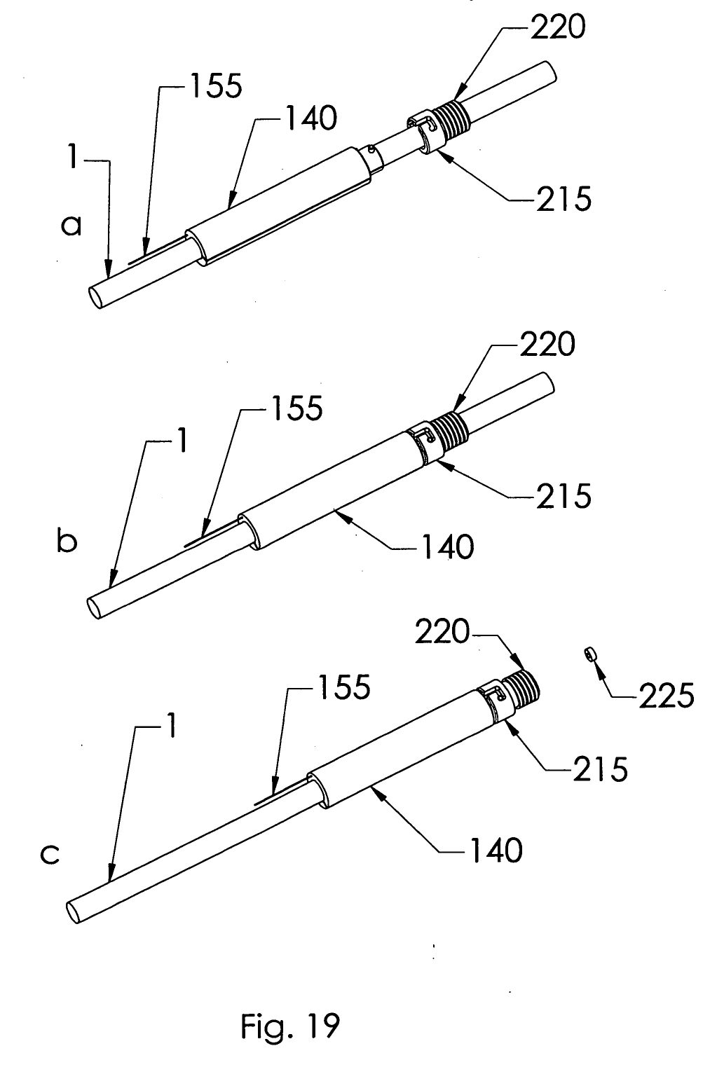

FIG. 19 illustrates another embodiment of a pre-placed device with a bayonet joint. This depicted pre-placed device can be used to place ligatures around tissue and generally are known as band ligators. FIG. 19 shows a sequence of three perspective views detailing the assembly and use of the pre-placed band ligating device 215. FIG. 19a illustrates the pre-placed band ligating device 215 positioned on the endoscope 1. The pre-placed band ligating device 215 may be preloaded with one or more, two or more, three or more, four or more, five or more, or six or more elastic ligatures 220 that are stretched to a much larger perimeter than in their free state. In one embodiment, about six ligatures can be pre-loaded onto a pre-placed device. FIG. 19a also shows a section of the overtube 140 in position on the endoscope 1 and in close proximity to the pre-placed band ligating device 215. FIG. 19b shows the overtube 140 and pre-placed band ligating device 215 assembled together by actuation of the bayonet joint as previously described. FIG. 19c shows the pre-placed band ligating device 215 in a typical operational position near the distal end of the endoscope 1. The control wire 155 has been actuated by the endoscopist in order to advance a ligature 220 off the end of the pre-placed band ligating device 215. A deployed ligature 225 in its much smaller free state is shown.

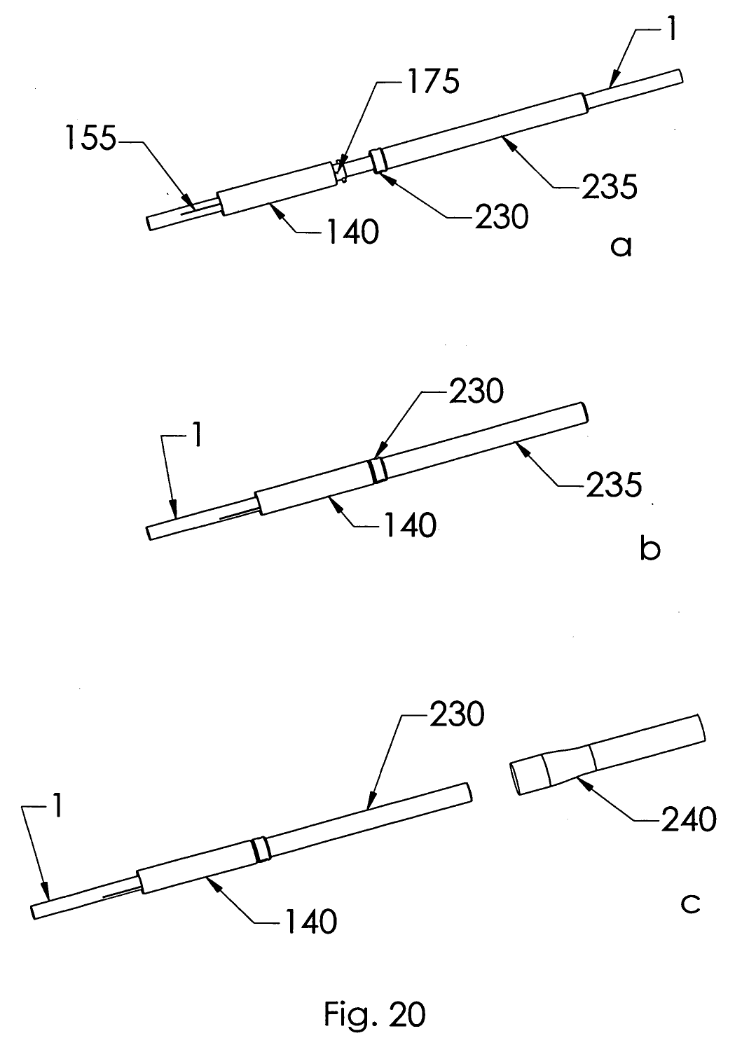

FIG. 20 illustrates another embodiment of a pre-placed device. In this depicted embodiment, the pre-placed device can be used to place expanding stents inside strictures, such as, without limitation, in the esophagus. FIG. 20 shows a sequence of three perspective views detailing the assembly and use of the pre-placed stent delivery device 230. FIG. 20a illustrates the pre-placed stent delivery device 230 positioned on the endoscope 1. The pre-placed stent delivery device 230 is preloaded with an expanding stent 235 that is constrained to a small perimeter by an over-wrapping thread or sheath. FIG. 20a also shows a section of the overtube 140 in position on the endoscope 1 and in close proximity to the pre-placed stent delivery device 230. Control wires 155 with hook ends 175 are also shown. The control wires can be used to initiate and control the expansion of the stent once properly positioned within a body cavity. With multiple control wires 155 it can be possible to more accurately control the expansion of the stent. For example, if two control wires 155 are used, expansion of the stent can be initiated from both its proximal and distal ends simultaneously or in sequence. This is in contrast to the current art in esophageal stent expansion in which expansion is only initiated from one end. Also in contrast to the current state of the art, placement of the stent can be done under endoscopic viewing rather than by fluoroscopic visualization. It is anticipated that much more accurate and rapid positioning of the stent at the stricture can be possible under endoscopic viewing. FIG. 20b shows the overtube 140 and pre-placed stent delivery device 230 assembled together by actuation of the bayonet joint as previously described. FIG. 20c shows the pre-placed stent delivery device 230 in a typical operational position near the distal end of the endoscope 1. The control wires 155 have been actuated by the endoscopist in order to deploy the expanding stent 235. As the stent is expanded, the endoscope 1, overtube 140 and pre-placed stent delivery device 230 are retracted from the stricture site. A deployed stent 240 in its expanded free state is shown in position at the stricture site.

It is to be understood that the present invention is not limited to the particular embodiments, materials, and examples described herein, as these can vary. It also is to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a receiving facsimile device” or “a document” is a reference to one or more receiving facsimile devices or documents and includes equivalents thereof known to those skilled in the art and so forth.

Unless defined otherwise, all technical terms used herein have the same meanings as commonly understood by one of ordinary skill in the art to which this invention belongs. Specific methods, devices, and materials are described, although any methods and materials similar or equivalent to those described herein can be used in the practice or testing of the present invention.

What is claimed is:

1. A device comprising a flexible tube and one or more open channels within the wall of said flexible tube wherein said tube further comprises a break along its length and the inner circumference of said tube is within about 0.5 mm of the outer perimeter of an endoscope and wherein said flexible tube is introduced onto said endoscope with the use of an introducer.

2. The device according to claim 1 wherein said one or more open channels within said wall of said flexible tube extend the length of said tube.

3. The device according to claim 1 wherein said lengthwise break can be closed.

4. An endoscope comprising an introducer for use with a device according to claim 1.

5. The device according to claim 1 wherein said one or more open channels are collapsed in their free state.

6. The device according to claim 1 wherein said flexible tube is segmented along its length with sections of semi-rigid and flexible materials.

7. The device according to claim 6 wherein said sections of semi-rigid and flexible materials alternate along said length of said flexible tube.

8. The device according to claim 1 wherein said device further comprises a clamp mechanism at or near the proximal end of said flexible tube.

9. The device according to claim 1 wherein one or more of said open channels house a control wire that terminates proximally in a control device.

10. The device according to claim 9 wherein the distal end of said control wire comprises one or more hooks.

11. The device according to claim 1 wherein one end and a portion of said flexible tube adjacent thereto have a perimeter that is reduced compared to the rest of said flexible tube.

12. The device according to claim 11 wherein said reduced perimeter portion of said flexible tube comprises one or more lugs.

13. A system comprising a first device comprising a flexible tube and one or more open channels within the wall of said flexible tube wherein said tube further comprises a break along its length and the inner circumference of said tube is within about 0.5 mm of the outer perimeter of an endoscope and a second device wherein said second device comprises a tube with an inner circumference of within about 0.5 mm of said outer perimeter of said endoscope.

14. The system according to claim 13 wherein said one or more open channels within said wall of said first device extend the length of said first device.

15. The system according to claim 13 wherein said lengthwise break of said first device can be closed.

16. An endoscope comprising an introducer for use with a system according to claim 13.

17. The system according to claim 13 wherein said one or more open channels within said wall of said first device are collapsed in their free state.

18. The system according to claim 13 wherein said flexible tube of said first device is segmented along its length with sections of semi-rigid and flexible materials.

19. The system according to claim 18 wherein said sections of semi-rigid and flexible materials alternate along said length of said flexible tube of said first device.

20. The system according to claim 13 wherein said first device further comprises a clamp mechanism at or near its proximal end.

21. The system according to claim 13 wherein one or more of said open channels within said wall of said first device house a control wire that terminates proximally in a control device.

22. The system according to claim 21 wherein the distal end of said control wire comprises one or more hooks.

23. The system according to claim 22 wherein said first device and said second device can be linked.

24. The system according to claim 23 wherein on said first device, one end and a portion of said first device adjacent thereto have a perimeter that is reduced compared to the rest of said first device.

25. The system according to claim 24 wherein said reduced perimeter portion of said first device fits within the perimeter of said second device.

26. The system according to claim 25 wherein said reduced perimeter portion of said first device further comprises one or more lugs and one end and a portion adjacent thereto of said second device comprise one or more slots configured to receive said one or more lugs.

27. The system according to claim 26 wherein said one or more lugs and said one or more slots can securely link said first device and said second device.

28. The system according to claim 27 wherein said link is in the form of a bayonet joint.

29. The system according to claim 23 wherein said second device comprises an actuator member.

30. The system according to claim 26 wherein said second device comprises an actuator that comprises an eyelet and wherein when said first device and said second device are linked, said hook of said wire engages said eyelet of said actuator member and said wire can be used to control said second device.

31. The system according to claim 13 wherein said second device is adapted to perform a function selected from the group consisting of providing a space to adjust the distance of the endoscope tip to tissue, detecting changes in tissue, providing a space to aspirate and hold tissue, providing a medium to enhance optics, deploying a stent, deploying a ligating element, deploying a cutting instrument with diathermy, deploying a cutting instrument without diathermy, deploying an anchoring element, deploying a joining element, and deploying tissue ablative energy.

32. A device comprising a tubular portion with an inner perimeter of within about 0.5 mm of the outer perimeter of an endoscope wherein said device comprises an actuator.

33. The device according to claim 32 wherein said actuator comprises an eyelet.

34. The device according to claim 32 wherein said device is adapted to perform a function near the end of an endoscope selected from the group consisting of providing a space to adjust the distance of the endoscope tip to tissue, detecting changes in tissue, providing a space to aspirate and hold tissue, providing a medium to enhance optics, deploying a stent, deploying a ligating element, deploying a cutting instrument with diathermy, deploying a cutting instrument without diathermy, deploying an anchoring element, deploying a joining element, and deploying tissue ablative energy.

35. A method comprising:
performing an endoscopic procedure on a patient with an endoscope, wherein said endoscope is associated with an overtube, wherein said overtube comprises a flexible tube and one or more open channels within the wall of said flexible tube wherein said overtube further comprises a break along its length and the inner circumference of said overtube is within about 0.5 mm of the outer perimeter of said endoscope and wherein said overtube is introduced onto said endoscope with the use of an introducer.

36. The method according to claim 35 wherein said one or more open channels within said wall of said flexible tube extend the length of said tube.

37. The method according to claim 35 wherein said lengthwise break can be closed.

38. The method according to claim 35 wherein said endoscope comprises said introducer.

39. The method according to claim 35 wherein said one or more open channels are collapsed in their free state.

40. The method according to claim 35 wherein said overtube is segmented along its length with sections of semi-rigid and flexible materials.

41. The method according to claim 40 wherein said sections of semi-rigid and flexible materials alternate along said length of said overtube.

42. The method according to claim 35 wherein said overtube further comprises a clamp mechanism at or near its proximal end.

43. The method according to claim 35 wherein one or more of said open channels house a control wire that terminates proximally in a control device operated by a person involved in the performing of said endoscopic procedure.

44. The method according to claim 43 wherein the distal end of said control wire comprises one or more hooks.

45. The method according to claim 35 wherein one end and a portion of said overtube adjacent thereto have a perimeter that is reduced compared to the rest of said overtube.

46. The method according to claim 45 wherein said reduced perimeter portion of said overtube comprises one or more lugs.

47. The method according to claim 35 wherein said performing of said endoscopic procedure further comprises associating a pre-placed device with said endoscope wherein said pre-placed device comprises a tube with an inner circumference of within about 0.5 mm of said outer perimeter of said endoscope and said associating occurs through placing said pre-placed device around said outer perimeter of said endoscope.

48. The method according to claim 47 wherein said performing of said endoscopic procedure further comprises linking said overtube and said pre-placed device.

49. The method according to claim 48 wherein one or more of said open channels house a control wire that terminates proximally in a control device operated by a person involved in the performing of said endoscopic procedure.

50. The method according to claim 49 wherein the distal end of said control wire comprises one or more hooks.

51. The method according to claim 50 wherein said linking occurs through the use of one or more lugs and one or more slots to form a bayonet joint.

52. The method according to claim 47 wherein said pre-placed device comprises an actuator member.

53. The method according to claim 50 wherein said pre-placed device comprises an actuator that comprises an eyelet and wherein when said overtube and said pre-placed device are linked, said hook of said wire engages said eyelet of said actuator member and said wire can be used to control said pre-placed device.

54. The method according to claim 47 wherein one aspect of said endoscopic procedure comprises using said pre-placed device to perform a function selected from the group consisting of providing a space to adjust the distance of the endoscope tip to tissue, detecting changes in tissue, providing a space to aspirate and hold tissue, providing a medium to enhance optics, deploying a stent, deploying a ligating element, deploying a cutting instrument with diathermy, deploying a cutting instrument without diathermy, deploying an anchoring element, deploying a joining element, and deploying tissue ablative energy.

55. A method comprising:
assembling an endoscopic device wherein said endoscopic device comprises an endoscope and an overtube, wherein said overtube comprises a flexible tube and one or more open channels within the wall of said flexible tube wherein said tube further comprises a break along its length and the inner circumference of said tube is within about 0.5 mm of the outer perimeter of said endoscope and wherein said assembling comprises placing said overtube around the outer perimeter of said endoscope with the use of an introducer.

56. The method according to claim 55 wherein said one or more open channels within said wall of said flexible tube extend the length of said tube.

57. The method according to claim 55 wherein said lengthwise break can be closed.

58. The method according to claim 55 wherein said endoscope comprises an introducer for use in said placing of said overtube.

59. The method according to claim 55 wherein said one or more open channels are collapsed in their free state.

60. The method according to claim 55 wherein said flexible tube is segmented along its length with sections of semi-rigid and flexible materials.

61. The method according to claim 60 wherein said sections of semi-rigid and flexible materials alternate along said length of said flexible tube.

62. The method according to claim 55 wherein said overtube further comprises a clamp mechanism at or near its proximal end and said method further comprises clamping said mechanism after said placing.

63. The method according to claim 55 wherein one or more of said open channels house a control wire that terminates proximally in a control device.

64. The method according to claim 63 wherein the distal end of said control wire comprises one or more hooks.

65. The method according to claim 55 wherein said assembling further comprises associating said endoscope of said endoscopic device with a pre-placed device, said pre-placed device comprising a tube with an inner circumference of within about 0.5 mm of said outer perimeter of said endoscope and wherein said associating comprises placing said pre-placed device around said outer perimeter of said endoscope.

66. The method according to claim 65 wherein said method further comprises linking said overtube and said pre-placed device.

67. The method according to claim 65 wherein one or more of said open channels house a control wire that terminates proximally in a control device.

68. The method according to claim 67 wherein the distal end of said control wire comprises one or more hooks.

69. The method according to claim 66 wherein said linking occurs through the use of a bayonet joint using one or more lugs on said overtube and one or more slots on said pre-placed device.

70. The method according to claim 65 wherein said pre-placed device comprises an actuator member.

71. The method according to claim 68 wherein said pre-placed device comprises an actuator that comprises an eyelet and wherein when said overtube and said pre-placed device are linked, said hook of said wire engages said eyelet of said actuator member and said wire can be used to control said pre-placed device.

72. The method according to claim 65 wherein said pre-placed device is adapted to perform a function selected from the group consisting of providing a space to adjust the distance of the endoscope tip to tissue, detecting changes in tissue, providing a space to aspirate and hold tissue, providing a medium to enhance optics, deploying a stent, deploying a ligating element, deploying a cutting instrument with diathermy, deploying a cutting instrument without diathermy, deploying an anchoring element, deploying a joining element, and deploying tissue ablative energy.
